(19) United States
(12) Patent Application Publication
(10) Pub. No.: US 2026/0167118 A1
(43) Pub. Date: Jun. 18, 2026

(54) HAND-HELD UNIT FOR FEEDING AND RELEASING AN IMPLANT

(71) Applicant: OPTIMED MEDIZINISCHE INSTRUMENTE GMBH, Ettlingen (DE)

(72) Inventors: Achim ZIPSE, Baden-Baden (DE); Jonas FRANKE, Karlsruhe (DE)

(21) Appl. No.: 18/716,924

(22) PCT Filed: Dec. 2, 2022
(86) PCT No.: PCT/EP2022/084205

§ 371 Date: Jun. 5, 2024

(30) Foreign Application Priority Data
Dec. 6, 2021 (DE) 10 2021 132 098.4

(51) Int. Cl. B60R 11/02; A61F 2/95

(52) U.S. Cl. CPC B60R 11/02; A61F 2/95

(57) ABSTRACT

A hand-held unit serves to feed an implant, in particular a stent, and to release the implant, and comprises a housing that has a housing opening and a connection section for attaching an outer catheter layer of a catheter to the housing and for guiding a release element from an interior of the housing into the catheter so that the release element is movable relative to the outer catheter layer, and an actuation wheel that is rotatably supported in the housing and that can be actuated through the housing opening in order to effect a rotary movement of the actuation wheel, wherein the actuation wheel has a receiver for a pulling element so that the actuation wheel exerts a pulling force on the release element via the pulling element during a rotary movement of the actuation wheel in a release direction, wherein the receiver is designed as helical to roll up the pulling element by means the receiver.

The invention relates to a hand-held unit for feeding and releasing an implant.

Such hand-held units are in particular used to insert an implant, for example a stent, stent graft, covered stent, filter, etc., into the body of a patient and in particular to place it in a blood vessel. In this respect, the implant is first brought to a specific position within the blood vessel by means of a catheter connected to the hand-held unit. Once the correct position for the implant has been reached, the implant is inserted at this position. With self-expanding implants or stents, the insertion can take place by removing a sleeve or a jacket surrounding the implant, also designated as a middle catheter or middle catheter layer, from the implant so that the implant can expand with respect to its periphery, supports the blood vessel and remains in the release position. Self-expanding implants or stents can consist of a shape memory alloy, for example. Other implants or stents can, for example, be expanded by balloon dilatation so that they remain in place.

It is an object of the present invention to improve the operability of a hand-held unit.

This object is satisfied by a hand-held unit having the features of one of the independent claims. Advantageous embodiments are the subject of the dependent claims.

The hand-held unit according to the invention serves to feed an implant, in particular a stent, and to release the implant and comprises a housing and an actuation wheel rotatably supported in the housing. The housing has a housing opening and a connection section. The connection section serves to attach an outer catheter layer of a catheter to the housing and to guide a release element from an interior of the housing into the catheter so that the release element is movable relative to the outer catheter layer. The actuation wheel can be actuated through the housing opening to effect a rotary movement of the actuation wheel and has a receiver for a pulling element so that the actuation wheel exerts a pulling force on the release element via the pulling element during a rotary movement of the actuation wheel in a release direction, wherein the receiver is designed as helical to roll up the pulling element by means of the receiver.

According to the invention, the hand-held unit is in particular used to introduce the implant by means of the catheter into a—in particular human—body of a patient, and indeed substantially into a blood vessel of the patient, to position it there and then to release it so that the implant remains at the location at which it was positioned. The catheter in particular has an inner catheter layer, a middle catheter layer and an outer catheter layer, wherein the middle catheter layer is movable relative to the inner and the outer catheter layer and the implant is received between the inner and the middle catheter layer before the release. The term “release” refers to the removal of the implant from the catheter so that the implant can remain at the position in the body or blood vessel of the patient. As a rule, the middle catheter layer surrounding the implant is retracted, i.e. moved away from the implant towards the hand-held unit, while the implant is held radially inside, in particular by a form fit, against the movement of the middle catheter layer on the inner catheter layer. After the retraction of the middle catheter layer, the implant can expand with respect to its periphery. With self-expanding implants, this happens automatically as soon as the middle catheter layer releases the implant. Other, non-self-expanding implants are e.g. expanded by inflating a balloon. According to the invention, the retraction of the middle catheter layer takes place by the pulling force exerted by the pulling element on the release element and the thereby produced movement of the release element. In this respect, the release element usually corresponds to the middle catheter layer, but it is also conceivable that the release element is a separate element that transmits the pulling force to the middle catheter layer.

The actuation wheel can be moved through the housing opening. This includes, on the one hand, that a part of the actuation wheel itself extends through the housing opening so that this part of the actuation wheel is outside the housing and can be manually actuated, in particular rotated, by a user there. On the other hand, this can also be understood in that the actuation wheel is indeed arranged completely inside the housing, but a part of the actuation wheel can be acted on from outside through the housing opening so that the actuation wheel can be moved.

The hand-held unit can have a blocking apparatus for blocking a rotary movement of the actuation wheel against the release direction so that the actuation wheel can only be rotated in the release direction. The blocking apparatus can in particular act as a freewheel or one-way clutch on the actuation wheel.

The blocking apparatus preferably has at least one pawl for engaging into a toothing of the actuation wheel. In particular, two or more pawls can also be provided at the blocking apparatus that can be arranged at equal spacings from one another at the blocking apparatus to engage into the toothing of the actuation wheel in each case.

The actuation wheel can be provided with a pocket in which the blocking apparatus is arranged in a rotationally fixed manner with respect to the housing and is preferably arranged coaxially with the actuation wheel. Due to the pocket, the blocking apparatus can engage into the toothing from the inside.

The housing can in particular be provided with a support projection at which the blocking apparatus is at least partly supported, with the support projection blocking a rotation of the blocking apparatus relative to the housing. The projection can, for example, be angular, for instance quadrangular, in particular rectangular or square, in cross-section. The blocking apparatus can then be provided with a corresponding recess so that the blocking apparatus can be placed onto the projection, while forming a form fit effective in the peripheral direction of the blocking apparatus, to support the blocking apparatus non-rotatably with respect to the housing.

The hand-held unit can furthermore have a locking apparatus for locking the actuation wheel. Locking here corresponds to fixing the actuation wheel in the release direction and the opposite direction. In particular, when positioning the implant, it can be advantageous to lock the actuation wheel and to only cancel the locking when the implant is correctly positioned in the body of the patient and is to be released there.

The locking apparatus preferably has a locking arm for engaging into a toothing of the actuation wheel. In particular, it can advantageously be the same toothing into which the blocking apparatus also engages. For this purpose, the locking arm and blocking apparatus are configured such that they can be simultaneously inserted into the pocket of the actuation wheel and can engage into the toothing side by side there.

The locking apparatus can have a spring element by which it is preloaded to lock the actuation wheel or is released from the locking. In particular, the spring element can be configured as a projecting wing whose free end comes into contact with a housing wall so that the locking apparatus is brought into engagement with the toothing by a spring force acting on said locking apparatus. To cancel the locking, the locking apparatus must therefore be moved out of engagement with the toothing against the spring force.

In an advantageous embodiment, the housing has a second housing opening through which the locking apparatus can be actuated in order to release the actuation wheel against the preloading force. The locking apparatus can be actuated, for example, by means of a button or by pressing on a surface provided for this purpose. When actuated, the locking apparatus is pushed out of the toothing against the spring force so that the locking of the actuation wheel is cancelled. The housing is preferably provided with a snap-in element into which the actuated locking apparatus latches so that the locking of the actuation wheel remains permanently cancelled until the locking apparatus is released from the latch connection by the practitioner or operator.

The locking apparatus can in particular be designed in two parts and have a locking arm and a release lever that is formed separately from the locking arm and can be actuated from outside the housing. The locking arm is then configured, when the release lever is actuated by a release mechanism, to be moved in order to release the actuation wheel. A part of the release lever can extend through the second housing opening to be able to be actuated from the outside. The release mechanism can, for example, be achieved by two slanted contact surfaces, with one contact surface each being provided at the release lever and the locking arm. When the release lever is actuated, the contact surfaces come into contact and slide along one another, wherein a movement of the locking arm out of the locking of the actuation wheel is brought about.

The radius of the receiver preferably becomes larger in the course of the rolling up. In a plan view, the helical receiver preferably extends in a spiral shape from the inside to the outside. Since the radius of the receiver is variable, the ratio of a movement of the actuation wheel in the release direction to a wound-up length of the pulling element changes, said pulling element, for example, being designed as a flexible pulling element, in particular as a cord. The retraction of the pulling element can thereby be adapted to the respective treatment situation, depending on whether a particularly careful movement of the pulling element—and thus of the middle catheter layer surrounding the implant—is required.

The hand-held unit preferably has a deflection device for the pulling element. The deflection device and the connection section are preferably arranged at oppositely disposed ends of the housing. In this respect, it is advantageous if the housing is elongate so that a certain path length can be achieved for the pulling at the release element. It is therefore advisable to arrange the deflection device at the end or in the region of the end of the housing that is disposed opposite the connection section. The deflection device can in particular be designed as cylindrical or hollow cylindrical so that the pulling element is guided around and deflected while contacting a part of the lateral surface of the cylinder.

The release element can be provided with a connector at which the pulling element engages; the connector can in particular be glued to the release element or the middle catheter layer. A pulling force of the pulling element can thereby lead to a pulling force on the release element and thus also to a movement of the release element.

In an advantageous embodiment, the housing comprises a guide for guiding an inner catheter layer, also called an inner catheter, out of the interior of the housing, with the guide and the connection section preferably being arranged at oppositely disposed ends of the housing. A Luer lock connection can be fastened to the end of the inner catheter and to the housing with a form fit. The fastening of the Luer lock connection to the inner catheter can in particular be designed so that a flushing of the inner catheter and an introduction of a guide wire through the inner catheter are made possible.

The release element can, for example, extend centrally with the actuation wheel so that a leading around the actuation wheel is obsolete.

For better handling, the housing can be provided with depressions for the fingers of an operator or practitioner. The housing then lies particularly comfortably in the hand and can be held securely and steadily during treatment.

The invention will be described schematically and by way of example with reference to embodiments and to the drawings. It is shown therein:

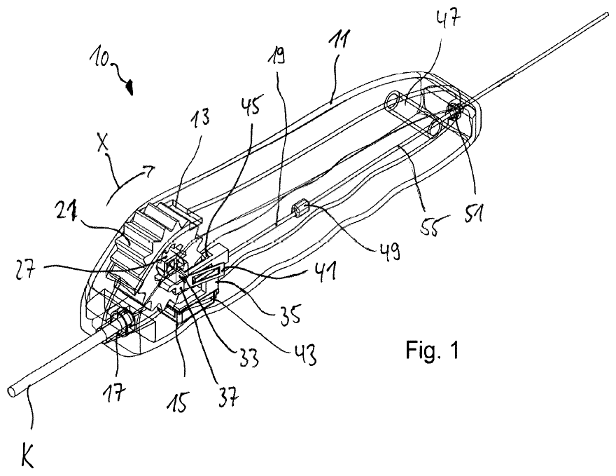

FIG. 1 a perspective view of a hand-held unit according to an embodiment example;

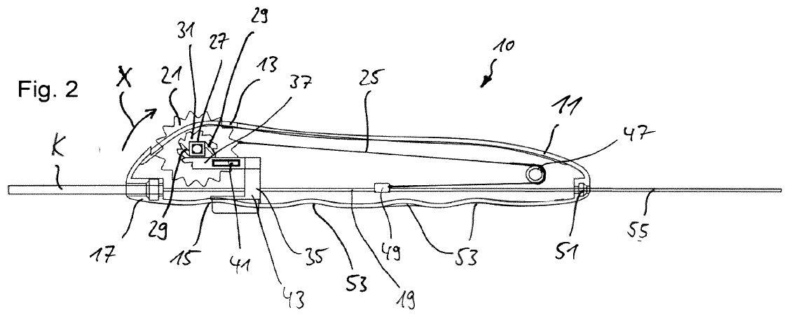

FIG. 2 a longitudinal sectional view of the hand-held unit of FIG. 1;

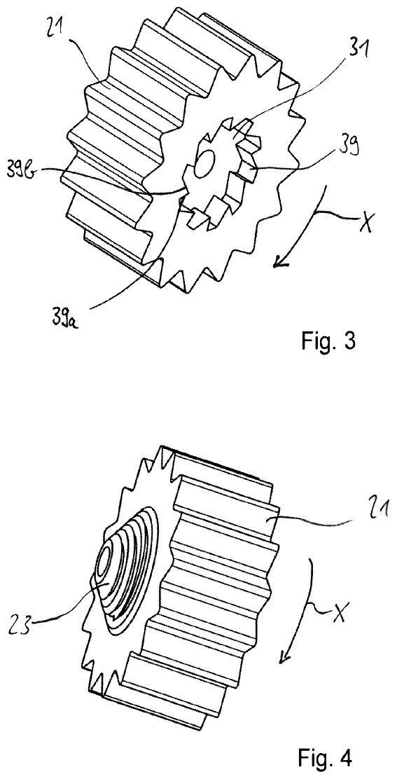

FIG. 3 a perspective view of the actuation wheel of FIG. 1; and

FIG. 4 a further perspective view of the actuation wheel of FIG. 1.

FIGS. 1 and 2 show a hand-held unit 10 comprising a housing 11 whose walls are shown transparent so that the interior of the housing 11 is clearly visible.

The housing 11 has a housing opening 13 and a connection section 17 to which a catheter K is attached with which a stent, not shown, is inserted by means of the hand-held unit 10 into the blood vessel of a patient, is positioned there and is then released to remain in the blood vessel.

A release element 19 is guided through the connection section 17 out of the housing interior into the catheter K and is moveable relative thereto, in particular to an outer catheter layer and an inner catheter layer. In the present embodiment example, the release element 19 corresponds to the middle catheter layer that surrounds the stent before the release. The stent is therefore received between the inner and middle catheter layer before the release. Alternatively, it is also conceivable that the release element 19 is formed separately from the middle catheter layer, but can exert a pulling force on it.

If a pulling force acting on the release element 19 in the housing interior causes a movement of the release element 19 away from the outer catheter layer of the catheter K, the middle catheter layer surrounding the implant and corresponding to the release element 19 in the present example is removed from the implant in the catheter K. The implant can thereby expand to remain in the blood vessel.

The force acting on the release element 19 is generated by a pulling element 25, shown exclusively in FIG. 2, that is connected to the release element 19 by means of a connector 49. The pulling element 25 can in particular be a cord and is guided from the connector 49 to a cylindrical deflection device 47, there around the lateral surface of the deflection device 47 and then from the deflection device 47 to a receiver 23 of an actuation wheel 21. The connector 49 is furthermore connected to an inner catheter layer 55 that is guided out of the housing 11 by a guide 51.

The actuation wheel 21 shown separately in FIGS. 3 and 4 is rotatably supported in the housing 11.

Furthermore, the actuation wheel 21 is provided with a receiver 23 that is designed as helical. The helical receiver 23 is designed such that the radius of the receiver 23 increases in the course of the rolling up. At its oppositely disposed side, on the other hand, the actuation wheel 21 has a pocket 31 whose side walls are provided with a toothing 39. The teeth of the toothing 39 are asymmetrical and each have a steep flank 39a against the release direction X and a flat flank 39b in the release direction X.

The actuation wheel 21 furthermore has a tooth-like contour at its outer periphery as a contact surface for the fingers of a practitioner or operator so that a practitioner or operator does not slip off the actuation wheel 21 when releasing an implant and operates the actuation wheel at a non-slip surface. To further optimize the handling of the hand-held unit 10, it is advisable to provide the housing 11 with depressions 53 at its lower side for the further fingers of the operator so that the housing 11 fits easily in the hand.

A part of the actuation wheel 21 projects through the upper housing opening 13 so that the actuation wheel 21 can be actuated (rotated) from outside the housing 11. In this respect, a rotary movement of the actuation wheel 21 in a release direction X causes the pulling element 25 to be rolled up in the receiver 23 and thus a pulling force to be transmitted to the release element 19 via the connector 49.

To prevent an unintentional rotation of the actuation wheel 21 against the release direction X, the hand-held unit 10 in the present example is provided with a blocking apparatus 27 that has two pawls 29 arranged at oppositely disposed sides of the blocking apparatus 27. In this respect, the blocking apparatus 27 can also have only one pawl 29 or more than two pawls 29. In the present example, however, it is advisable to provide two pawls 29 since the blocking apparatus 27 can thus be placed onto a support projection 33 provided at the housing 11 such that said blocking apparatus 27 is located in the pocket 31 of the actuation wheel 21 and a locking apparatus 35 can nevertheless also be inserted into the pocket 31, which will be described in detail later. In the present example, the support projection 33 is arranged coaxially to the axis of the actuation wheel 21 and is square in cross-section; however, other cross-sections, in particular angular cross-sections, are also conceivable. However, it is essential that the blocking apparatus 27 cannot be rotated relative to the housing 11.

The pawls 29 engage into the toothing 39, wherein the pawls 29 together with the steep flanks 39a block a rotation of the actuation wheel 21 against the release direction X and a rotation of the actuation wheel 21 in the release direction X is enabled due to the flat flanks 39b. The blocking apparatus 27 thus represents a freewheel or a one-way clutch for the actuation wheel 21.

Furthermore, the actuation wheel 21 can be completely locked by the locking apparatus 35, i.e. such that neither a rotation of the actuation wheel 21 against the release direction X nor a rotation of the actuation wheel 21 in the release direction X is possible. In the present embodiment example, the locking apparatus 35 is formed in two parts and has a locking arm 37 and a release lever 43 formed separately from the locking arm 37.

The locking arm 37 has a spring element 41 that projects laterally from the locking arm 37 and that preloads the locking arm 37 against the housing 11 so that the locking arm 37 is located in the pocket 31 due to the preloading force, engages into the toothing 39 and locks the actuation wheel 21.

The release lever 43 extends through a lower housing opening 15, which is disposed opposite the upper housing opening 13, so that the release lever 43 can be actuated through the lower housing opening 15, namely by pushing and moving said release lever upwards. The release lever 43 then latches in at a snap-in element of the housing 11 so that the release lever 43 remains in the actuated position. The actuation of the release lever 43 via a release mechanism 45 causes the locking arm 37 to be moved out of the pocket 31 and to release the toothing 39 and the actuation wheel 21. The locking is then cancelled. The release mechanism 45 is brought about by one slanted contact surface each at the release lever 43 and the locking arm 37. When the release lever 43 is actuated, the contact surfaces come into contact and slide along one another, wherein a movement of the locking arm 37 out of the locking of the actuation wheel 21 is brought about. The release mechanism 45 thus converts the upward movement of the release lever 43 into a lateral movement of the locking arm 37.

### REFERENCE NUMERAL LIST

10 hand-held unit
11 housing
13 housing opening
15 housing opening
17 connection section
19 release element
21 actuation wheel
23 receiver
25 pulling element
27 blocking apparatus
29 pawl
31 pocket
33 support projection
35 locking apparatus
37 locking arm
39 toothing
41 spring element
43 release lever
45 release mechanism
47 deflection device
49 connector
51 guide
53 depression
55 inner catheter layer
K catheter
X release direction

1-16. (canceled)

17. A hand-held unit for feeding an implant and for releasing the implant, said hand-held unit comprising
a housing that has a housing opening and a connection section for attaching an outer catheter layer of a catheter to the housing and for guiding a release element from an interior of the housing into the catheter so that the release element is movable relative to the outer catheter layer, and
an actuation wheel that is rotatably supported in the housing and that can be actuated through the housing opening in order to effect a rotary movement of the actuation wheel, wherein the actuation wheel has a receiver for a pulling element so that the actuation wheel exerts a pulling force on the release element via the pulling element during a rotary movement of the actuation wheel in a release direction, wherein the receiver is designed as helical to roll up the pulling element by means of the receiver.

18. The hand-held unit according to claim 17,
further comprising a blocking apparatus for blocking a rotary movement of the actuation wheel against the release direction.

19. The hand-held unit according to claim 18,
wherein the blocking apparatus has at least one pawl for engaging into a toothing of the actuation wheel.

20. The hand-held unit according to claim 18,
wherein the actuation wheel is provided with a pocket in which the blocking apparatus is arranged in a rotationally fixed manner with respect to the housing.

21. The hand-held unit according to claim 18,
wherein the housing is provided with a support projection at which the blocking apparatus is at least partly supported, with the support projection blocking a rotation of the blocking apparatus relative to the housing.

22. The hand-held unit according to claim 17,
further comprising a locking apparatus for locking the actuation wheel.

23. The hand-held unit according to claim 22,
wherein the locking apparatus has a locking arm for engaging into a toothing of the actuation wheel.

24. The hand-held unit according to claim 22,
wherein the locking apparatus has a spring element and is preloaded by the spring element to lock the actuation wheel or is released from the locking.

25. The hand-held unit according to claim 24,
wherein the housing has a second housing opening through which the locking apparatus can be actuated in order to release the actuation wheel against the preloading force.

26. The hand-held unit according to claim 22,
wherein the locking apparatus is designed in two parts and has a locking arm and a release lever that is formed separately from the locking arm and can be actuated from outside the housing, with the locking arm being configured, when the release lever is actuated by a release mechanism, to be moved in order to release the actuation wheel.

27. The hand-held unit according to claim 17,
wherein a radius of the receiver becomes larger in the course of the rolling up.

28. The hand-held unit according to claim 17,
further comprising a deflection device for the pulling element.

29. The hand-held unit according to claim 17,
wherein the release element is provided with a connector at which the pulling element engages.

30. The hand-held unit according to claim 17,
wherein the housing comprises a guide for guiding the release element out of the interior of the housing.

31. The hand-held unit according to claim 17,
wherein the release element extends centrally with the actuation wheel.

32. The hand-held unit according to claim 17,
wherein the housing is provided with depressions for the fingers of an operator.

33. The hand-held unit according to claim 20,
wherein the blocking apparatus is arranged coaxially with the actuation wheel.

34. The hand-held unit according to claim 25,
wherein the housing is provided with a snap-in element into which the actuated locking apparatus latches.

35. The hand-held unit according to claim 28,
wherein the deflection device and the connection section are arranged at oppositely disposed ends of the housing.

36. The hand-held unit according to claim 30,
wherein the guide and the connection section are arranged at oppositely disposed ends of the housing.
